(19) United States
(12) Patent Application Publication
(10) Pub. No.: US 2026/0102115 A1
(43) Pub. Date: Apr. 16, 2026

(54) DAVIDSON AIRWAY FUNCTION AND NASAL EVALUATION (DAFNE) SCORE INTERPRETATION AND CALCULATION METHODS FOR CLINICAL APPLICATION AND ARTIFICIAL INTELLIGENCE IN THE TREATMENT OF BREATHING DISORDERS AT SLEEP AND AWAKE USING OBJECTIVE NASAL MEASUREMENTS

(71) Applicant: Karen Parker Davidson, Gambrills, MD (US)

(72) Inventor: Karen Parker Davidson, Gambrills, MD (US)

(21) Appl. No.: 17/803,199

(22) Filed: May 12, 2023

(51) Int. Cl. A61B 5/00; G16H 15/00; G16H 50/30; G16H 50/70

(52) U.S. Cl. CPC A61B 5/4818; A61B 5/7221; A61B 5/7267; A61B 5/7465; G16H 15/00; G16H 50/30; G16H 50/70

(57) ABSTRACT

The invention is a novel process and method for using objective nasal measurements to include nasal flow, nasal resistance, nasal cycle, nasal volume, mean cross sectional area, deviations of data, the amount of mucosal involvement, the amount of effort of nasal breathing in the sitting, standing, and supine positions, mouth breathing, tongue posture involvement, data, age, gender, and race/ethnicity to predict the extent of disease, amount of disease progression, the severity of sleep apnea prior to a sleep test, best treatment option, and direct artificial intelligence for treating sleep and breathing disorders. According to the invention, the algorithmic method is accurate and dependable in assessing severity of OSA, nasal dysfunction, obstruction, and appropriate recommendations for treating sleep and breathing conditions from infants to adults and at certain milestones: baseline, treatment in progress, and post treatment to include the stability of outcomes short term and long term.

### CROSS-REFERENCE TO RELATED APPLICATIONS

This current application claims priority to U.S. Provisional Application No. 63/347,519 entitled “DAFNE Score Software and Calculation Methods” to Karen Parker Davidson, filed on May 31, 2022, the disclosure of which his hereby incorporated by reference in its entirety.

### BACKGROUND OF THE INVENTION

The concept of measuring nasal flow limitations and resistance has been discussed in the medical community since 1894; however, 129 years later, as of 2023, there is no interpretation software platform or algorithm for nasal measurements and artificial intelligence (AI) for clinical decision making in the office or for telemedicine. There is a need to define variable nasal function and flow that may be more important in pathophysiology of sleep and breathing problems than a fixed obstruction, or physical blockage.

More than 1 billion people around the world have a breathing disorder while sleeping with a number value of 5 (AHI) to detect the presences of sleep apnea, and greater than 20% deal with nasal obstruction. More than 13.6 million people in the United States deal with one nasal breathing problem costing $94.9 billion dollars in healthcare resources.

The industry standard has been to use the apnea-hypopnea index (AHI) as the benchmark for therapy and improvement of sleep apnea; however, nasal function and flow have not been considered. There is mounting evidence of a nasal endotype that predisposes people to systemic disease from improper breathing long before it is present. It has been proven that low nasal flow limitations and high resistance are present in the predisposition of sleep and breathing problems before clinical detection

The use of pulse sound waves and manometry is highly detective in measuring nasal flow, resistance, and function but lacks medical interpretation software which refers to algorithmic programs or applications designed to assist in the interpretation and translation of medical information between different languages.

Nasal resistance measurements can help diagnose and monitor nasal airway disorders, evaluate the effectiveness of treatments, and guide surgical interventions if necessary.

Treatment options for nasal resistance may include medications to reduce inflammation, nasal dilators to improve airflow, or oral appliances, surgical procedures to correct structural abnormalities.

A medical interpretation algorithm refers to a set of computational instructions or steps designed to process and analyze medical data or information with the aim of providing meaningful interpretations or insights. These algorithms leverage various techniques, including statistical analysis, machine learning, and natural language processing, to interpret medical data and assist healthcare professionals in making informed decisions.

These tools are specifically tailored to meet the unique needs and challenges of medical communication, ensuring accurate and efficient interpretation in healthcare settings. Here are some key features and benefits of medical interpretation software:
Subjective data has been a standard of care when evaluating the severity of disease and effectiveness of treatment; however, the accuracy remains in question as opinion lacks accuracy.
Objective numeric data has been used to quantify disease disposition, potential, presence, absence, or improvement as well as the best treatment option.
By integrating multiple data sources and utilizing machine learning algorithms, this novel invention method allows for a comprehensive and data-driven interpretation of nasal function and flow.
DAFNE Score and calculations methods enhance diagnostic accuracy, provide personalized insights, and supports evidence-based decision-making in the evaluation and management of nasal conditions.
Nasal measurements refer to various anatomical and physiological measurements related to the nose. These measurements are often taken for medical, anthropological, or aesthetic purposes and provide valuable information about the structure and function of the nose.
Medical and dental interpretation software capabilities refer to computer programs, algorithms, or applications designed to assist in the interpretation and translation of medical information between different languages.
Medical and dental methods and processes encompass the systematic approaches, protocols, and procedures followed in the field of medicine and dentistry for various purposes, including diagnosis, treatment, prevention, and research.
Medical and dental methods and processes ensure consistency, reliability, and standardization in healthcare delivery.

These tools are specifically tailored to meet the unique needs and challenges of communication, ensuring accurate and efficient interpretation in healthcare settings. Here are some key features and benefits of medical interpretation software:
Airway measurements refer to the assessment and quantification of various parameters related to the respiratory system's anatomy and function. These measurements help evaluate the size, patency, and function of the airways, aiding in the diagnosis and management of respiratory conditions. Here's an overview of the background of airway measurements:
Airway measurements play a crucial role in diagnosing and monitoring respiratory conditions, evaluating treatment response, and assessing the impact of interventions in clinical and research environments.

These measurements help healthcare professionals tailor appropriate management plans and optimize patient care. Interpretation of airway measurements should consider individual patient characteristics, age, gender, and reference values established for specific populations.

Airway measurements are necessary and dependent on normal parameters based on age, gender, and race/ethnicity to predict the extent of disease, best treatment option, and artificial intelligence for treating sleep and breathing disorders.

The user enters patient demographic data and measurements obtained from nasal measurement devices into DAFNE that generates an output describing the amount of disease progression and nasal function during active breathing.

The output educates and guides the healthcare provider how to diagnose, choose the best treatment, and collaborate with other providers.

Data is saved on a cloud for analysis of pattern recognition for AI. According to the invention, the algorithmic process is accurate and reliable in assessing severity of OSA, nasal dysfunction, obstruction, and appropriate recommendations for treating sleep and breathing conditions from infants to adults and at certain milestones: baseline, treatment in progress, and post treatment to include the stability of outcomes short term and long term.

Use of the interpretation algorithm will allow better understanding of airway diseases involving nasal function and structure.

The present invention aims to address these problems and provide a solution that is capable of determining what the data means, how to use it for treatment decisions, and how to monitor progress and changes in patient care through several key aspects of the algorithm such from infants to adults.

This method is the first available interpretation process in the world and consists of:
Data Collection: Gather a comprehensive set of data related to nasal function, including patient demographic information, medical history, symptom profiles, nasal endoscopy images or videos, nasal airflow measurements, acoustic rhinometry data, and subjective patient-reported outcomes.
Data Preprocessing: Prepare the collected data for analysis by cleaning and organizing it. This step involves removing any noise or outliers, standardizing data formats, and ensuring data consistency across diverse sources.
Feature Extraction: Extract relevant features from the collected data that provide meaningful information about nasal function. These features can include nasal airflow patterns, nasal cavity dimensions, nasal resistance values, and subjective symptom scores. Advanced image processing techniques can also be applied to extract features from nasal endoscopy images or videos.
Integration of Data: Combine the several types of data collected from various sources into a unified dataset. This integration allows for a comprehensive analysis of nasal function, considering multiple aspects and measurements.
Machine Learning Modeling: Utilize machine learning algorithms, such as decision trees, support vector machines, or neural networks, to train predictive models. These models learn patterns and relationships within the data and can predict or classify nasal function based on the extracted features.
Model Training and Validation: Split the dataset into training and validation sets. The training set is used to train the machine learning models, while the validation set is used to evaluate their performance and fine-tune the models if necessary. Cross-validation techniques can also be employed to ensure robustness and avoid overfitting.
Interpretation and Analysis: Once the models are trained and validated, they can be used to interpret nasal function based on new input data. The models can predict nasal function parameters, classify nasal conditions or abnormalities, and provide insights into the factors influencing nasal function, such as anatomical variations, nasal congestion, or airway obstruction.
Clinical Decision Support: The interpreted nasal function results can be used as a clinical decision support tool, assisting healthcare professionals in diagnosing and managing nasal conditions. The results can guide treatment planning, including the selection of medications, surgical interventions, or other therapeutic options.
Artificial Intelligence for Nasal Air Function and Flow: Artificial Intelligence (AI) has shown immense potential in the analysis and interpretation of nasal air function and flow.

This invention and method are the introductory platform, foundation, and premise to AI for nasal and airway measurement and consists of:
Image Analysis: AI algorithms can be employed to analyze nasal geometry by training deep learning models on a large dataset of numeric data from annotated tracings and wave forms. AI can assist in automatic detection and classification of nasal anatomical structures, such as turbinates, septum, and nasal valves. This analysis can provide insights into the amount and location of nasal airway obstruction or deviations.
Airflow Simulation and Prediction: AI techniques, specifically mean flow limitations and nasal resistance, can simulate and predict nasal airflow patterns. AI algorithms can integrate patient-specific data, resistance values, and percentage of obstruction, nasal flow limitations and variations, to create personalized airflow models. This can aid in the identification of regions of airflow turbulence, areas of nasal obstruction, dentofacial anomalies, septal deviation, postural affects, involvement of the central and autonomic nervous systems, and the impact of surgical interventions, oral appliance therapy, myofunctional therapy, and other therapies, nasal dilatation, heart rate variability, or nasal treatments on airflow. Furthermore, the measured flow limitations emulate the correlation between the measurement and the AHI, RDI, and ODI found in a sleep study.
Diagnostic Support: AI can assist in the diagnosis of nasal conditions by analyzing various data sources, including patient symptoms, medical history, imaging studies, and nasal airflow measurements. Machine learning algorithms can learn patterns from a large dataset of patient data and provide diagnostic support to healthcare professionals. This can help in identifying nasal disorders like chronic rhinosinusitis, nasal polyps, or nasal valve collapse.
Treatment Planning: AI algorithms can contribute to personalized treatment planning for nasal conditions. By considering patient-specific data and clinical guidelines, AI can help determine the most appropriate treatment options, such as medications, nasal sprays, or surgical interventions. AI can also assist in predicting treatment outcomes and optimizing treatment strategies based on individual patient characteristics.
Remote Monitoring: AI-powered devices and sensors can be used for remote monitoring of nasal air function and flow using peak nasal flow measurements. These devices can collect real-time data on nasal airflow, and other relevant parameters. AI algorithms can analyze this data, identify abnormalities or trends, and provide feedback to patients and healthcare providers. This enables remote monitoring of nasal conditions and early detection of changes or exacerbations, and when to contact the provider
Predictive Analytics: AI can leverage data from large patient cohorts to develop predictive models for nasal air function and flow. By analyzing patterns in patient data, AI algorithms can predict disease progression, treatment response, or the likelihood of complications. This can assist in making informed decisions regarding treatment planning and patient management.

The development and deployment of this invention and interpretation algorithms for nasal function and flow is highly detective of disease, is validated, able to integrate with clinical workflows, designed to show effective simulation of a sleep study prior to a sleep test, and underwent consideration of ethical and legal considerations through extensive research.

The invention method and algorithms maintain patient privacy with nonidentifying information, are intended to augment clinical decision-making and should always be used in conjunction with the expertise and judgment of healthcare professionals.

### SUMMARY OF THE INVENTION

Methods for clinical application and artificial intelligence (AI) in the treatment of nasal breathing dysfunction and breathing disorders at sleep and awake using objective nasal measurements involve the use of advanced techniques to assess and manage respiratory conditions.

These methods aim to provide objective and accurate measurements of nasal function, allowing for personalized and targeted treatment approaches. Additionally, AI algorithms are employed to analyze the collected data and provide valuable insights for diagnosis, treatment planning, and monitoring of breathing disorders.

Key components of these nasal measurement methods may include objective nasal measurements.

Current FDA approved objective diagnostic tests for nasal airway measurements are rhinomanometry, peak nasal inspiratory flow (PNIF), and acoustic rhinometry,

Various techniques are used to objectively measure nasal function, such as acoustic rhinometry, rhinomanometry, or nasal endoscopy. These measurements assess nasal airflow, resistance, and anatomical factors, providing quantitative data for analysis. Data on nasal measurements, patient characteristics, symptoms, and treatment outcomes are collected and analyzed. This involves the integration of multiple data sources to identify patterns and relationships between nasal measurements and breathing disorders. AI algorithms enhance diagnostic accuracy and aid in personalized treatment planning.

The invention addresses the objective nasal measurements and AI analytics that contribute to individualized treatment planning. The data help guide the selection of appropriate interventions, such as nasal medications, surgical procedures, or continuous positive airway pressure (CPAP) therapy. Additionally, AI algorithms can be utilized for monitoring treatment progress and adjusting therapeutic approaches as needed.

These methods aim to integrate objective nasal measurements and AI analysis into routine clinical practice. By providing clinicians with objective data and decision support tools, these methods enhance the accuracy and efficiency of diagnosis, treatment, and monitoring of breathing disorders.

Interpretation algorithm and software capabilities are lacking for airway measurements, specifically on objective nasal measurements.

The absence of interpretation guidelines and processes are absent in the global market leaving the end user unaware of what the date output means, how to interpret the data, understand the normative values, and how treatment options will be affected based on the measurements and output information from DAFNE.

The DAFNE score software and calculation methods are a validated process and platform to answer the essential tools in the clinical management of breathing disorders during sleep and wakefulness that answers the need for interpretation guidance and education.

The use of objective nasal measurements, age, gender, and race/ethnicity in the calculation of the DAFNE score allows for accurate and reliable assessment of nasal function, aiding in the diagnosis and treatment of a range of respiratory conditions at certain milestones of therapy to include baseline, treatment in progress, and post treatment to include the stability of outcomes short term and long term.

Artificial intelligence (AI) is also increasingly being used in the treatment of breathing disorders, allowing for more personalized and precise care.

By incorporating AI algorithms and machine learning in the form of pattern recognition, the analysis of objective nasal measurements within DAFNE will assist healthcare providers the ability to obtain more detailed and nuanced information about an individual's nasal function, which can guide treatment decisions and improve outcomes.

The DAFNE score software and calculation methods in clinical practice highlights the potential benefits of incorporating AI and machine learning into the assessment and treatment of breathing disorders.

By utilizing these tools and techniques, healthcare providers can better understand the underlying causes of respiratory conditions, develop more effective treatment plans, and improve the overall quality of care for individuals with breathing disorders while collaborating with other healthcare providers.

With more than a billion people around the world suffering from nasal obstruction and sleep apnea, the quantifiable data used in the algorithm of DAFNE will be able to identify and stratify more patients for earlier intervention with the appropriate treatment.

With more than 13.6 million people in the United States affected by at least one nasal breathing disorder accounting for 94.4 billion dollars in utilized healthcare resources, in addition to the excessive amount of lost productivity in the workplace, the methodology behind the algorithm and process will be able to identify and stratify more patients.

### BRIEF DESCRIPTION OF DRAWINGS

FIG. 1 is the DAFNE Model Framework that shows the method of structured approach or building, implementing, or evaluating the invention method.

FIG. 1 illustrates how to solve the problem or the question of nasal flow limitations and obstruction that further identifies the goals, constraints, and desired outcomes of the algorithm suitable for the specific problem and data characteristics.

FIG. 1 illustrates how the data is collected and prepared which is necessary to train and evaluate the data from various sources and transforming it into a suitable format; to identify and extract meaningful features from the raw data that can be used as input variables for the model. This may involve data transformation, scaling, encoding categorical variables, and creating new derived features.

FIG. 1 illustrates how the method will train the selected model using the prepared dataset that involves optimizing the model's parameters or weights to minimize the error or loss function. The training process typically involves splitting the data into training and validation sets and iteratively adjusting the model's parameters using various optimization techniques.

FIG. 1 illustrates how the model assesses the performance of the trained method using appropriate evaluation metrics. This may involve metrics such as accuracy, precision, and recall of nasal flow and function. Cross-validation techniques can be used to evaluate the model's performance on multiple subsets of the data with pattern recognition and statistical analysis of the measurements across all populations.

FIG. 2 illustrates the methods and calculations using nasal flow and pressure across a pressure gradient during active breathing a step-by-step process from signing into the account to pattern recognition for AI. The shows how the method refers to a set of computational steps and calculations used to analyze and interpret nasal airflow and resistance measurements obtained through a form of mamometry that measures the resistance to airflow through the nasal passages, providing objective data on nasal function. The method begins with the collection of nasal manometry data. This involves inserting small tubes or sensors into the patient's nostrils to measure the airflow and pressure one side at a time for a unilateral test or in the mouth for a bilateral test. The method calculates nasal resistance by dividing the pressure drop across the nasal cavity by the corresponding airflow rate. This calculation provides an objective measure of nasal obstruction or resistance. Based on the calculated nasal resistance values, the method can classify the degree of nasal obstruction or resistance into different categories such as normal, mild, moderate, or severe. The specific cutoff values for these classifications may vary based on clinical presentation. The method generates a report summarizing the results and interpretation. It may provide additional information or recommendations based on the interpretation, such as suggesting further diagnostic tests, treatment options, or referrals to specialists. The method is designed to integrate into clinical practice and should provide clinicians with meaningful and actionable insights to aid in diagnosis, treatment planning, or monitoring of nasal conditions.

FIG. 3 illustrates the methods and calculations using acoustic sound pulse waves in a step-by-step process from signing into the account to pattern recognition for AI.

FIG. 4 illustrates the methods and calculations using nasal air flow in a step-by-step process from signing into the account to pattern recognition for AI.

FIG. 5 illustrates the decision tree of patient identification based on nasal resistance measured in Pascals, also known as a unit, used to measure internal pressure and mechanical stress, from signing into the account to pattern recognition for AI. Is the patient experiencing symptoms of nasal congestion or obstruction? If Yes, measure for transnasal pressure changes. What level is the nasal resistance? Is there a structural abnormality seen in the tracing? Is there extreme effort in active breathing? What body position is the test? Is a nasal provocation test needed? If Yes, what was the change in measurements? Include the Age: Evaluate for age-related changes in nasal resistance. Body position: Assess the impact of body position on nasal airflow. Environmental factor: Consider exposure to allergens, irritants, or pollutants. The exact tested measurements are not available for public view. Each treatment will be indicated based on the level of transnasal pressure, flow, and nasal resistance. Each treatment will show improvement at a certain level of change in the transnasal pressure change as a biomarker measured in Pascals. The decision tree describes how much improvement is seen for each treatment in infants, children and adults.

FIG. 6 illustrates the scoring considerations affecting the data output of the method. Nasal cycle, position changes, and decongestion changes will affect the treatment options, disease progression, and changes and are part of the treatment considerations.

FIG. 7 illustrates the method of interpretation using the PNIF algorithm designed to interpret and analyze Peak Nasal Inspiratory Flow (PNIF) measurements and pattern recognition for AI in the office or remotely through any aspect of telemedicine or teledentistry. PNIF is a commonly used measurement in assessing nasal airflow and function. The method begins by collecting PNIF measurements from the patient. PNIF measurements are typically obtained using a handheld device called a peak flow meter, specifically designed for nasal measurements. The patient is instructed to perform a forceful inhalation through the device, and the peak inspiratory flow rate is recorded. The method establishes specific threshold values to differentiate between normal and abnormal PNIF measurements. These thresholds can be determined based on amount is nasal flow, age, and gender. Based on the normalized PNIF values and the determined thresholds, the method interprets the PNIF measurements. It classifies the measurements into categories such as normal nasal airflow, mild, moderate, or severe nasal obstruction, or other relevant classifications based on the specific purpose of the algorithm. The method generates a report summarizing the interpretation of the PNIF measurements. It provides additional information or recommendations based on the interpretation, such as suggesting further diagnostic tests, treatment options, or referrals to specialists.

FIG. 8 illustrates the method of interpretation of nasal flow in cubic centimeters per second (ccm/s) that involves analyzing the measured flow rates and comparing them to established reference values or normal ranges that determines the amount of obstruction broken down in 20% increments, and how it correlated to the AHI, RDI, and ODI seen in sleep studies. Nasal flow limitations make sleep apnea more likely to appear before an apneic event as the body needs time to acclimate to the change. Nasal obstruction created from nasal flow limitations, which can result from various factors such as deviated septum, nasal polyps, or allergies, can contribute to sleep apnea. When nasal airflow is obstructed, individuals may compensate by breathing through their mouths, which can lead to an increased risk of sleep apnea events. Increased nasal resistance, often measured using techniques like rhinomanometry, can indicate nasal airflow limitations. Decreased nasal flow limitations may require increased effort to breathe through the nose, potentially contributing to sleep apnea severity. Nasal flow limitations can affect the effectiveness of Continuous Positive Airway Pressure (CPAP) therapy, which is a common treatment for sleep apnea, and Oral Appliance Therapy (OAT). If the nasal airflow is significantly restricted. It may be challenging for individuals to tolerate hypoglossal nerve stimulation, or CPAP or OAT therapy to achieve adequate air pressure levels to maintain airway patency during sleep. Nasal flow limitations typically interact with other factors that contribute to sleep apnea severity, such as obesity, anatomical abnormalities, or impaired muscle tone. The presence of nasal flow limitations can exacerbate the effects of these factors, leading to more severe sleep apnea. The method is able to show how the measured nasal flow rates in relation to the patient's baseline or normal flow are affecting treatment and improvement of airway disease via nasal breathing in the pediatric and adult populations. through the mouth, can also influence the relationship between nasal flow limitations and sleep apnea severity.

FIG. 9 illustrates how the method uses effective simulation of data output and compares the amount of obstruction and flow limitations to AHI Prior to a sleep study. The comparison of the testing output and method algorithm underlines the functionality of the DAFNE output by identifying patients predisposed to sleep and breathing disorders, intolerant for CPAP and OAT, and treatment progression. The sensitivity of the test and correlation to the method allows for early intervention and diagnosis before comorbid conditions happen.

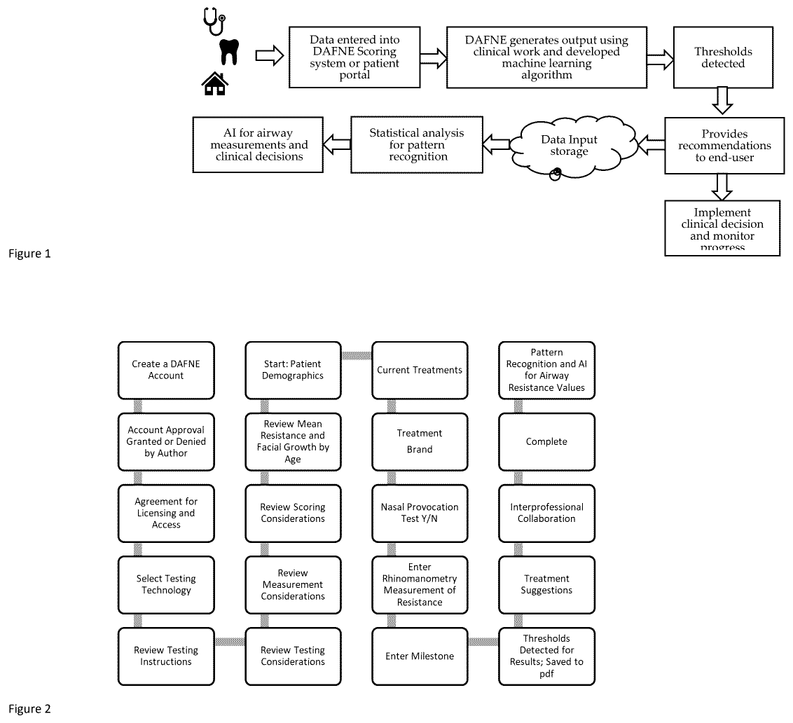

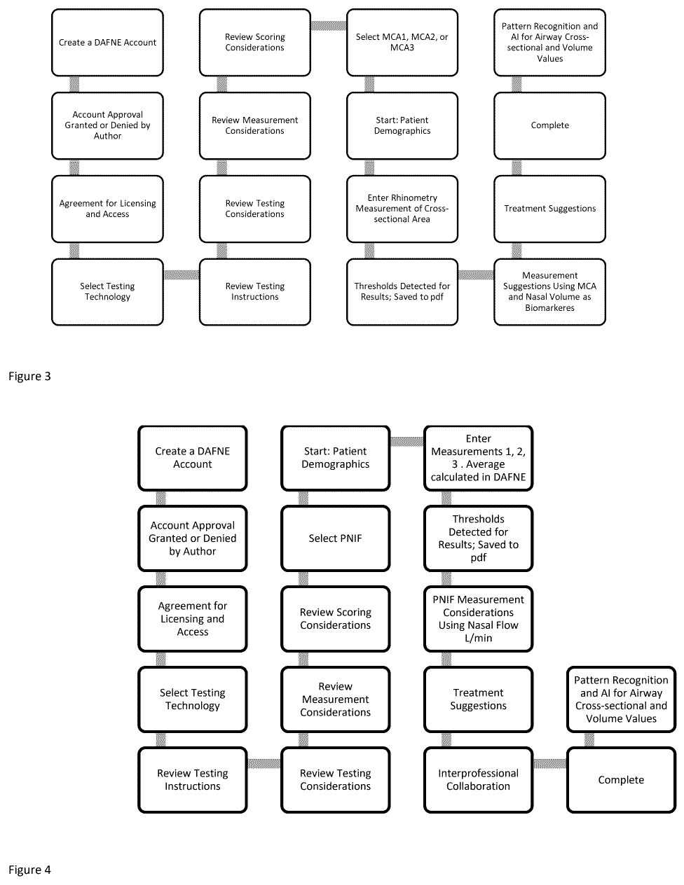

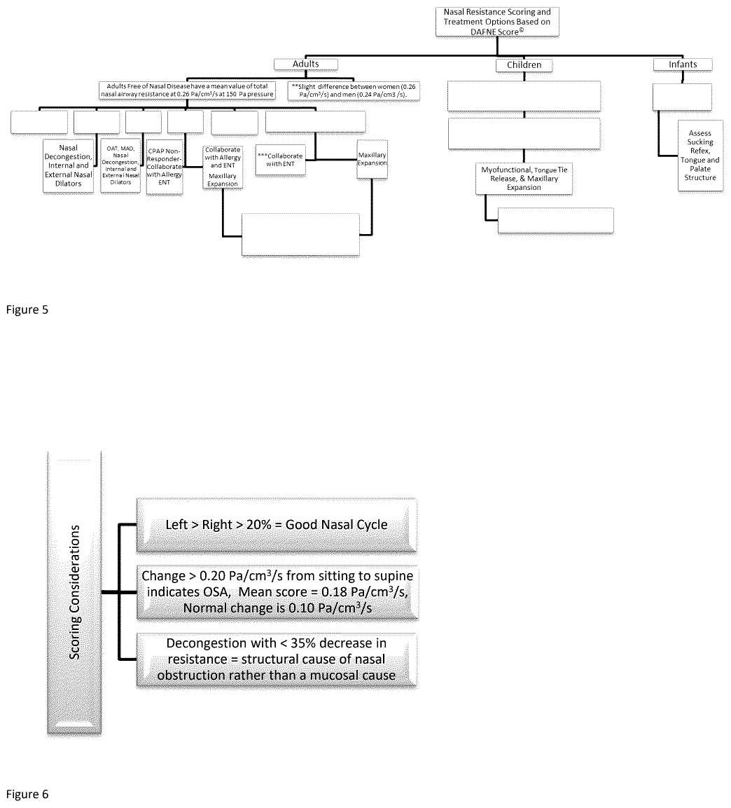

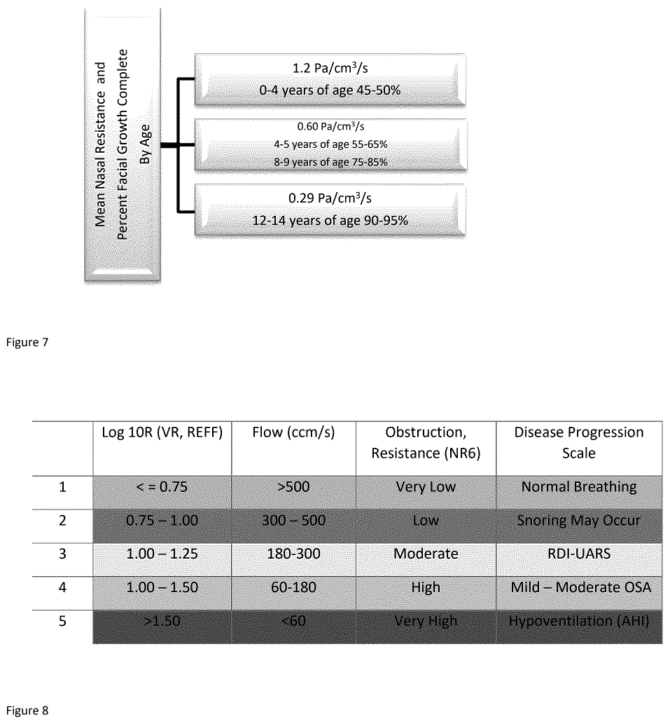

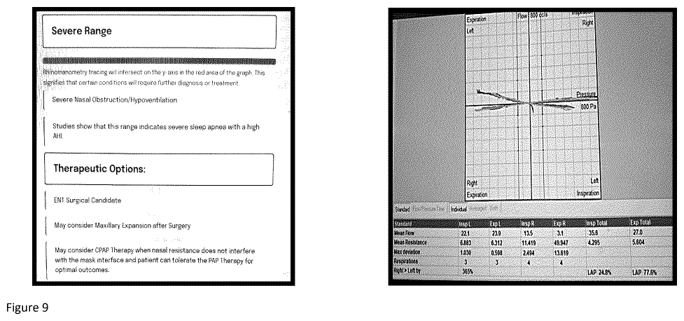

### DETAILED DESCRIPTION OF THE INVENTION

The present invention discloses an innovative airway measurement interpretation method that enables accurate and comprehensive assessment of nasorespiratory function, flow, and nasal airway characteristics. The method utilizes advanced algorithms and data analysis techniques to interpret the data obtained from various airway measurement techniques, providing valuable insights into the condition of the respiratory system.
Data Acquisition: The airway measurement interpretation method starts with the acquisition of data from multiple airway measurement techniques, such as spirometry, rhinometry, rhinomanometry. These techniques capture parameters such as nasal volumes, airflow limitations, airway resistance, and compliance.
Data Processing: The acquired data undergoes threshold detection preprocessing steps, and aligning the data obtained from different measurement techniques.
Feature Extraction: Relevant features are extracted from the preprocessed data to capture key aspects of nasorespiratory function and airway characteristics. These features may include nasal geometry, peak inspiratory flow rate (PIFR), airway resistance, and other derived parameters.
Algorithm Development: Advanced algorithms are developed to analyze the extracted features and provide meaningful interpretations. These algorithms may utilize machine learning techniques, pattern recognition, or mathematical models to identify patterns, abnormalities, or specific nasorespiratory conditions based on the collected data.
Reference Comparison: The interpreted data is compared with established reference values or normative data sets specific to age, sex, height, and other relevant factors. This comparison enables the determination of the deviation from normal ranges and helps in diagnosing respiratory disorders or assessing the severity of nasal airway abnormalities.
Integrated Analysis: The interpretation method integrates the information obtained from different airway measurement techniques, providing a comprehensive analysis of respiratory function. It considers the interrelationships between various parameters to provide a holistic understanding of the patient's airway health.
Visualization and Reporting: The interpreted results are visualized in a user-friendly format, such as graphs, charts, or summary reports. The visual representations highlight key findings, trends, and abnormalities, facilitating easy understanding and communication of the results to healthcare professionals and patients.
Clinical Decision Support: The interpretation method may include a clinical decision support system that offers recommendations based on the interpreted data. This may involve suggesting further diagnostic tests, treatment options, or referral to specialists based on the identified respiratory conditions or abnormalities.
Continuous Improvement: The airway measurement interpretation method is designed to be adaptable and continuously improved. It incorporates feedback from clinical users, incorporates new research findings, and updates the algorithms and interpretation criteria to enhance accuracy and clinical utility.

The invented airway measurement interpretation method revolutionizes the analysis and understanding of respiratory function and airway characteristics. Its integration of multiple measurement techniques, advanced algorithms, and comprehensive interpretation provides valuable insights for respiratory assessment, diagnosis, treatment planning, and monitoring. At least one specification heading is required.

1. A unique and novel method for detecting, diagnosing, and monitoring abnormal nasal function and flow limitations in the office or remotely with telemedicine and teledentistry consisting steps of:
a. Acquiring a test from the nose with a rhinomanometer, rhinometer, or peak nasal flow meter, sitting or supine
b. Entering and calculating the test result into the web-based program with software capabilities under the appropriate technology source
c. Filtering data by the algorithm of various nasal measurement thresholds
d. Assigning nasal measurement thresholds
e. Modeling by the algorithm for measurement threshold detection
f. Calculating nasal measurement variations at different nasal geometric segments, transnasal pressure changes as a biomarker, and nasal flow biomarkers
g. Assessing the patient's condition using nasal measurement biomarkers
h. Recommending treatment options provided to end users
i. Effective simulation of DAFNE output to apnea-hypopnea index (AHI) prior to a sleep study to show predisposition to sleep disorders
j. Generating an output from the method and saved as a file report
k. Collaboration pathway with other healthcare providers explained
l. Storing data input on a secured cloud for future data analysis
m. System data acquired overtime is analyzed for pattern recognition based on all variables combined within the algorithm
n. Artificial intelligence developed algorithms and DAFNE output can integrate patient-specific data, such as nasal geometry, resistance values, and anatomical variations, to create personalized treatment models.
o. Artificial intelligence is a powerful tool in healthcare that may improve patient care, enhance diagnostic abilities, and augment the management of breathing and sleep disorders

2. The method of claim 1, wherein the nasal measurement device comprises a PNIF nasal flowmeter, rhinomanometer, or acoustic rhinometry device.

3. The method of claim 1, wherein the preprocessing step includes filtering the nasal measurements, removing outlier values, and aligning data obtained from different nasal measurement techniques.

4. The method of claim 1, wherein the algorithm utilizes machine learning techniques, pattern recognition, or mathematical models to analyze the nasal measurements and derive the interpretive information.

5. A unique and novel method for interpreting nasal measurements comprising of:
a. Obtaining nasal measurements including nasal flow rate, nasal resistance, or other nasal parameters using a nasal measurement device.
b. Analyzing the preprocessed nasal measurements using an algorithm to derive interpretive information.
c. Comparing the interpretive information with established reference values or normative data specific to nasal measurements.
d. Generating an interpretive report indicating the deviation of the nasal measurements from the reference values or normative data.

6. The method of claim 2, wherein the interpretive information includes the classification of nasal measurements into categories indicating normal nasal function, mild deviation, moderate deviation, or severe deviation based on the comparison with reference values or normative data.

7. The method of claim 2, further comprising generating visual representations, such as graphs or charts, to present the interpretive information in a user-friendly format.

8. The method of claim 2, further comprising providing clinical recommendations based on the interpretive information, including suggestions for further diagnostic tests, treatment options, or referral to specialists.

9. The method of claim 2, wherein the interpretive report includes historical data of the nasal measurements for comparison and trend analysis.

10. The method of claim 2, wherein the interpretive report is customizable based on specific clinical requirements or preferences.

11. The method of claim 2, wherein the method is implemented in a computer system or software application to enable automated and efficient interpretation of nasal measurements.

12. The method of claim 2, wherein transitory computer-readable medium consists of a computer program with a set of executable instructions that when executed with a computer will perform the claimed functions.

13. A unique and novel method using a computer-implemented algorithm for assessing nasal flow and function contains the method comprising of:
a. Receiving nasal measurements including nasal airflow, nasal resistance, or other parameters related to nasal function.
b. Applying a mathematical model or machine learning techniques to the preprocessed nasal measurements to derive interpretive information.
c. Analyzing the interpretive information to assess nasal function based on established reference values or normative data specific to nasal function measurements.

14. The algorithm of claim 3, wherein the preprocessing step includes filtering the nasal measurements, removing outlier values, and aligning data obtained from different nasal function measurement techniques.

15. The algorithm of claim 3, wherein the mathematical model utilizes statistical analysis, pattern recognition, or other computational techniques to derive the interpretive information from the nasal measurements.

16. The algorithm of claim 3, wherein the interpretive information includes the classification of nasal function into categories indicating normal function, mild impairment, moderate impairment, or severe impairment based on the comparison with the reference values or normative data.

17. The algorithm of claim 3, further comprising generating visual representations, such as graphs or charts, to present the interpretive information in a user-friendly format.

18. The algorithm of claim 3, further comprising providing clinical recommendations based on the interpretive information, including suggestions for further evaluation, treatment options, or referral to specialists.

19. The algorithm of claim 3, wherein the interpretive information is updated in real-time as new nasal measurements are received, allowing for dynamic assessment of nasal function.

20. The algorithm of claim 3, wherein the algorithm is integrated into a software application or computer system for automated and efficient assessment of nasal function.

21. The algorithm of claim 3, wherein the interpretive information is stored on a cloud and can be accessed for historical analysis and trend monitoring of nasal function over time for AI

22. The algorithm of claim 3, further comprising a validation module that checks the quality and reliability of the nasal measurements before applying the mathematical model or machine learning techniques has been conducted and passed validation.
